SEQUENCE LISTING (2 sequences)

SEQ ID NO: 1 - DNA, 1698 bp, Yersinia pseudotuberculosis
atgaaagcatctgatgcaatagcgcatgtgttaagtgctaataatgtagattttggttttgaattgatcggtggaatgattgctcatcttgtggatagcatcaattcattagggaaaacaaaattagtttctgtacaccatgagcaggctgcagcatttgcagccggtggaatagctagagctacgggtaatgaaaaaataggtttagctttaggtactagtggacctggtgctacaaatttgataactggcattgcagattgttggttagatagttctccttgtatttttattacaggccaggtcaatactcatgaacttaagggggggcgtggtatccggcagcaaggttttcaagaattagatatagttagtattgttaaaagtataacaaagtatgcatatcagataaaaagcaaagatgaaataattccatgtttgcaaaatgcaatcgatatcgcaagaagtggtagacctgggcctgtgcttattgatatcccaatggatctgcaaagagctgaactagatgatagtataatcaattatcttaacaatgaaatagattctaatctgatatctaaaaaagattcatcatctatatttgatgagatttttagcttactagagcagtctaaacgacctgtatttttaattggtggtggtgcaatacacgaagaaaaattcaatctttggcaaaacaaaatttctgaaaataatataccccatgtcgccagtttaaaaggtgcggagaaaactttatcaataccaggctattttggtatggtcggtagctatggtacccgcactgctaattatgcagtacaaaatgctgacttggttattgttctaggtagccgtttagacgtaaggcagaccggtgctgacacgcaagattttgcaagaaacgcgaaaattgtccaaattgatattgatattgaacaattagataatagagtgaaatgtcaacttaatattaatgcatcatgtgaggatttcttttcttattatttacgttcgaggcaatgtaattcgtataactcttggttcctacaattagaaaacaattggactaaaaattttgtagatgaatatcaagatttaaaagtaagtccatttagccttttctccattctaaataatgcttttaatgataaaaatgtccattacgtaactgatgtcggcaacaatcagatgtgggcagcccattcattacgtttaggtgtagggcaagctattcataattctggaggcttgggtgcgatgggatttggcatcccaacagctattggtgtgtcatatgcgacaaactatcctgttgtagtaattactggggatggtggagctcaattgaatatccaagagctagatattatatctcgcgaaaatcgtcctatactaacaatagttttaaacaataattctttaggtatggtacgtgcttttcaggaaatgtattttgacggtagaaatgagtcaacatactggagtggatatagctcatcattctctaaaattgggaaagcatataatgttgagtcgtacactgtattaacagaatgtgactttttagaaaaagtcaatgtgtttattgataatccaaagccaatgcttattgaagttttgatggaagatgcaagagaatgtagaccaagattagcttttggtaacccaattgataaacaatttccctataaatag

SEQ ID NO: 2 - protein, 565 aa, Yersinia pseudotuberculosis
MKASDAIAHVLSANNVDFGFELIGGMIAHLVDSINSLGKTKLVSVHHEQAAAFAAGGIARATGNEKIGLALGTSGPGATNLITGIADCWLDSSPCIFITGQVNTHELKGGRGIRQQGFQELDIVSIVKSITKYAYQIKSKDEIIPCLQNAIDIARSGRPGPVLIDIPMDLQRAELDDSIINYLNNEIDSNLISKKDSSSIFDEIFSLLEQSKRPVFLIGGGAIHEEKFNLWQNKISENNIPHVASLKGAEKTLSIPGYFGMVGSYGTRTANYAVQNADLVIVLGSRLDVRQTGADTQDFARNAKIVQIDIDIEQLDNRVKCQLNINASCEDFFSYYLRSRQCNSYNSWFLQLENNWTKNFVDEYQDLKVSPFSLFSILNNAFNDKNVHYVTDVGNNQMWAAHSLRLGVGQAIHNSGGLGAMGFGIPTAIGVSYATNYPVVVITGDGGAQLNIQELDIISRENRPILTIVLNNNSLGMVRAFQEMYFDGRNESTYWSGYSSSFSKIGKAYNVESYTVLTECDFLEKVNVFIDNPKPMLIEVLMEDARECRPRLAFGNPIDKQFPYK

(19) United States
(12) Patent Application Publication
(10) Pub. No.: US 2011/0097766 A1
(43) Pub. Date: Apr. 28, 2011

(54) 1,2 - ADDITION OF CARBONYL COMPOUNDS USING THE ENZYME YERE

(75) Inventors: Michael Müller, Gundelfingen (DE); Patrizia Lehwald, Denzlingen (DE); Michael Richter, Freiburg (DE)

(73) Assignee: ALBERT-LUDGWIGS-UNIVERSITÄT FREIBURG, Freiburg (DE)

(21) Appl. No.: 12/993,070

(22) PCT Filed: May 14, 2009
(86) PCT No.: PCT/EP2009/055800

§ 371 Date: Nov. 16, 2010

(30) Foreign Application Priority Data
May 16, 2008 (EP) 08009054.1

(51) Int. Cl. C12P 17/06; C12P 7/26

(52) U.S. Cl. 435/125; 435/148

(57) ABSTRACT

A method is disclosed for forming a covalent bond between two groups having carbonyl radicals in accordance with the present reaction equation:

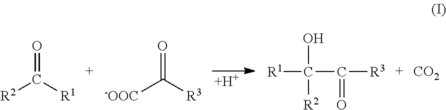

where
R¹ is hydrogen, a carboxyl group or a C₁-C₆ alkyl and
R² is an aromatic or heteroaromatic radical which may possibly be monosubstituted or disubstituted with halogen and/or a C₁-C₆ alkyl or is a substituent having the formula -A-B-C-D where A and C are each a C₁-C₆ alkyl, carbonyl or a covalent bond, B is a heteroatom such as oxygen and sulphur and D is an aliphatic radical having 1 to 6 carbon atoms, or an aromatic or heteroaromatic radical which may possibly be substituted, or where R¹ and R² together form a cyclic alkyl radical which may possibly be monosubstituted or disubstituted with a C₁-C₆ alkyl radical and/or with a hydroxy radical and which may possibly contain a heteroatom such as oxygen or sulphur, and R³ is hydrogen or a C₁-C₆ alkyl, wherein the reaction is catalysed by the enzyme YerE.

For the synthesis of enantiomeric pure natural products and active substances, it is often crucial for a large enantiomeric excess to be obtained in the individual steps of the synthesis. The present method relates to the reaction of two chemical groups having carbonyl radicals, namely aldehyde and ketone groups, in a benzoin condensation to form a carbon-carbon bond, the reaction being catalysed by a bacterial enzyme.

The benzoin condensation, in which aldehydes are converted into acyloins, is one of the reactions in organic chemistry of which knowledge goes back for the longest time. If one of the two aldehydes were replaced by a ketone, tertiary alcohols might be produced in this way. Because chiral tertiary alcohols are compounds which are of great interest for the synthesis of enantiomeric pure natural products and active substances, various chiral catalysts have been developed to enable the aldehyde-ketone benzoin reaction to be performed asymmetrically. However, the problem of intermolecular non-enzymatic asymmetrical acyloin condensation using ketones as acceptors has not as yet been solved. The reason for this is the lower carbonyl activity of ketones as compared with aldehydes, which lower carbonyl activity privileges an aldehyde-aldehyde benzoin reaction and hence the formation of acyloins.

It is true that the C—C-bonding of aldehydes in the 1,2-position (see benzoin condensation) using thiamine diphosphate (ThDP) dependent enzymes is possible with a good yield and a good enantiomeric excess, but it has not been possible to show evidence of an aldehyde-ketone benzoin reaction for conventional ThDP-dependent enzymes such for example as benzaldehyde lyase (BAL) from Pseudomonas fluorescens and pyruvate decarboxylase (PDC) from Saccharomyces cerevisiae.

It is an object of the present invention to provide a new method for forming a covalent bond between two groups having carbonyl radicals such that there is a defined stereo chemistry. The two groups having carbonyl radicals may preferably be located on different molecules and the reaction is then an intermolecular one. The two groups may, however, equally well be present on one and the same molecule and the reaction is then an intramolecular one.

With the method according to the invention, it is possible, using the thiamine diphosphate dependent enzyme YerE as a catalyst, to obtain by means of an intermolecular aldehyde-ketone benzoin reaction 1,2-addition products with an enantiomeric excess of, preferably >10:1, which corresponds to a percentage enantiomeric excess of >80%. In accordance with the invention the enantiomeric excess is at least 70% as a preference, preferably at least 75%, more preferably at least 80%, even more preferably 90%, even more preferably 95%, even more preferably 98%. Both cyclic and open-chain aliphatic ketones and also ketones having aromatic and heteroaromatic substituents may be used in this case as acceptor substrates. The protein YerE thus has a wide range of substrates.

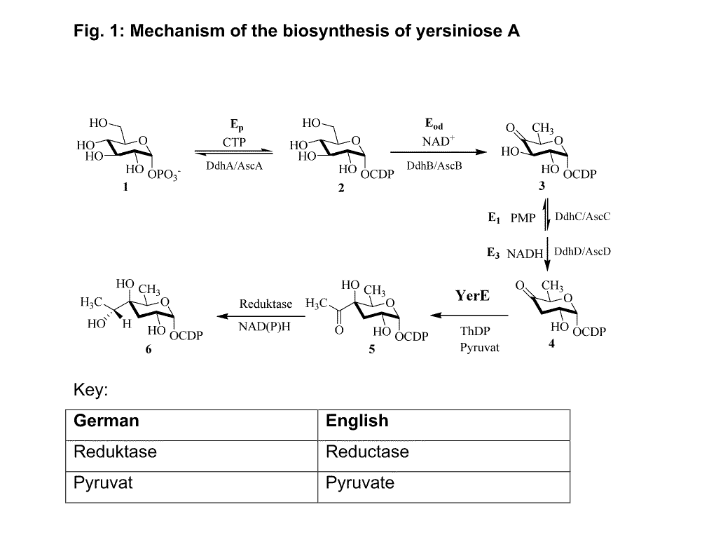

Liu et al. (J. Am. Chem. Soc. 1998, pp. 11796-11797) published in 1998 the mechanism of the biosynthesis of yersiniose A, a 3,6-didesoxyhexose which is a constituent of the lipopolysaccharides of Yersinia pseudotuberculosis O:VI. The ThDP-dependent enzyme which encodes by yerE catalyses in this case both the decarboxylation of pyruvate and also the acetyl transfer to an activated 3,6-didesoxy-4-keto-D-glucose. The enzyme YerE has been cloned by Liu et al. and purified by immobilised metal ion affinity chromatography. By gel filtration a molar weight of 117 kDa has been determined for the homodimer and a size of 63 kDa has been determined for each sub-unit. The function of the enzyme YerE in the biosynthesis of yersiniose A is shown in FIG. 1. To determine the activity of the enzyme, the substrate, activated 3,6-didesoxy-4-keto-D-glucose, was obtained, starting from CDP-D-Glucose, by the use of the purified enzymes E_{od}, E₁ and E₃ and evidence of its conversion by YerE into compound 5 was found by NMR spectroscopy. The reaction is also described by He, X. M. and Liu, H. W. in a review (Annual Review of Biochemistry, Vol. 71, 2002, pages 731-733).

The invention relates to an in vitro method so the part in question of the biosynthesis of yersiniose A in which the enzyme YerE catalyses both the decarboxylation of pyruvate and also the acetyl transfer to the activated 3,6-didesoxy-4-keto-D-glucose does not fall within the scope of the present invention. The starting compound in the method according to the invention is therefore not cytidine-diphosphate(CDP)-activated 3,6-didesoxy-4-keto-D-glucose.

Because, due to the CDP-activation, the physiological substrate is a very large molecule having a molar mass of 557 (see diagram),

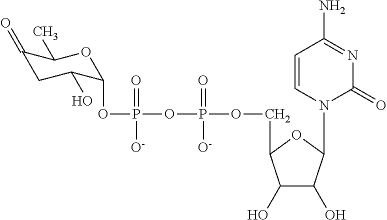

Diagram: Physiological acceptor substrate for YerE

the conversions that we demonstrate of very small cyclic acceptor substrates such as cyclohexanone having a molar mass of 98 or tetrahydro-2H-pyran-3-one having a molar mass of 100 (see diagram) are surprising. It could not in any way been foreseen that such small molecules could be stabilised and converted in the active centre of the enzyme.

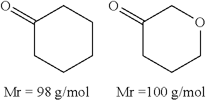

Diagram: Non-physiological cyclic acceptor substrates found for YerE

The present invention relates to a method for forming a covalent bond between two groups having carbonyl radicals in accordance with the present reaction equation:

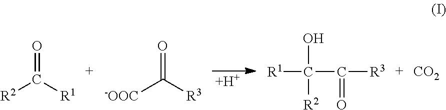

where

R¹ is hydrogen, a carboxyl group or a C₁-C₆ alkyl and

R² is an aromatic or heteroaromatic radical which may possibly be monosubstituted or disubstituted with a halogen and/or a C₁-C₆-alkyl or is a substituent having the formula -A-B-C-D where A and C are each a C₁-C₆ alkyl, a carbonyl or a covalent bond, B is a heteroatom such as oxygen or sulphur, and D is an aliphatic radical or an aromatic or heteroaromatic radical which may possibly be substituted,

or R¹ and R² together form a cyclic alkyl radical which may possibly be monosubstituted or disubstituted with a C₁-C₆ alkyl radical and/or with a heteroatom and which may possibly contain a heteroatom such as oxygen or sulphur, and

R³ is hydrogen or a C₁-C₆ alkyl, wherein the reaction is catalysed by the enzyme YerE.

Because the reaction which occurs naturally is not covered, R¹ and R² are not, in accordance with the invention, so defined that the molecule which takes part in the reaction is CDP-activated 3,6-didesoxy-4-keto-D-glucose.

The compound which carries the radicals R¹ and R² is preferably neither an activated nor a non-activated 3,6-didesoxy-4-keto-D-glucose. Furthermore, what are also preferably excluded as reactands in the reaction according to the invention are all 3,6-didesoxy-4-ketohexoses, whether activated or not. Also, the keto compound in the present reaction is preferably not a ketopyranose.

The enzyme YerE which is used in accordance with the invention was first described in the case of Yersinia pseudotuberculosis. What is preferably used in accordance with the invention is an enzyme produced by recombinant methods. The gene encoding for YerE was cloned from Yersinia pseudotuberculosis and is expressed in suitable host organisms. In a preferred embodiment, the gene encoding for YerE is incorporated in a suitable expression vector and expressed in a suitable host organism.

As part of the present invention, the gene encoding for YerE has been cloned and sequenced. The nucleic acid encoding for the protein is disclosed in the sequence listing as SEQ ID NO:1. However, within the scope of the present invention it is also possible for use to be made of enzymes similar to YerE which are encoded by a gene which has a similar sequence, although the enzymatic activity must be at least as great.

It is known to those skilled in the art that similar genes can also be isolated from other micro-organisms. The present invention therefore also relates to the use of enzymes similar to YerE which have, at the nucleotide level, a homology of at least 60%, preferably at least 80% and as a particular preference at least 90%, calculated over the entire length of SEQ ID NO:1. Commercially available programs can be used with standard settings to calculate the homology.

It is also known to those skilled in the art that the enzymatic activity of the enzyme can be improved by suitable mutations. Those amino acids which are responsible for the catalytic activity can be identified from a three-dimensional model of the tertiary structure which can be constructed with a suitable computer model. The enzymatic activity can be increased by the step-by-step replacement of individual amino acids.

SEQ ID NO:2 shows the amino acid sequence of the YerE enzyme. Within the scope of the present invention, use may also be made of enzymes which are very similar to SEQ ID NO:2. What are therefore preferably used within the scope of the present invention for the conversion according to the invention are enzymes which have a homology of at least 60%, preferably at least 70%, even more preferably at least 80% and, as a particular preference, at least 90% to the amino acid sequence having the identification number 2. The figures for homology relate in this case to the entire length of the protein shown in SEQ ID NO:2.

For the performance of the reaction, the enzyme YerE may be either highly purified or partly purified. Methods of purifying an enzyme are well known to the average person skilled in the art. Suitable tags coding for the enzyme YerE may for example be added to the sequence, which tags then interact with suitable purifying materials or adsorptive materials and make it possible for efficient and, in most cases, sufficient purification to take place in a single stage of purification. For use for the majority of purposes it is enough for a raw enzyme preparation to be used which preferably contains approximately 5 to 80% by weight, and as a particular preference 20 to 80% by weight, of YerE as percentages of the total protein content. However, for special embodiments it may also be advisable for the enzyme to be more highly purified so that the protein preparation contains 60 to 95% of YerE as a percentage of the total protein used. This is something which may be considered particularly when the enzyme is used in continuous processes and when, in this case, it is immobilised on a solid phase such for example as on chromatographic material, carrier beads or the like.

By the use of the enzyme YerE, it is possible in accordance with the invention to form a covalent bond between two chemical groups having carbonyl radicals. The two reactands in the above reaction equation (I) may be considered to be an acceptor substrate and a donor substrate.

In one embodiment, the acceptor is a compound having the formula

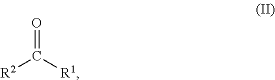

where the meaning of R¹ is hydrogen, a carboxyl group or a C₁-C₆ Alkyl and R² is an aromatic radical which may possibly be monosubstituted or disubstituted with a heteroatom and/or a C₁-C₆ alkyl.

The acceptor having the formula (II) may also be pyruvate. When this is the case, the meaning assumed by substituent R¹ is a carboxyl group and that assumed by substituent R² is a methyl group.

In a preferred embodiment, aldehydes are used as acceptors. When this is the case, the meaning assumed by substituent R¹ is hydrogen and the radical R² may have various meanings. In a preferred embodiment the meaning assumed by R² may be a monosubstituted or disubstituted aromatic radical. What may be involved in this case are aromatic 5-rings or 6-rings and condensed 5-rings and 6-rings, which may also contain heteroatoms such as oxygen, nitrogen or sulphur. Preferred examples of these aromatic rings are furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thioazole, benzothiazole, benzol, naphthalene, anthracene, pyridine, isoquinoline, pyrazine, quinoxaline, acridine, pyrimidine, quinazoline, pyridazine or cinnoline. A particular preference is phenyl. These aromatic radicals may be substituted with 1 to 2 radicals, the substituents preferably being halogens (F, Cl, Br, I), hydroxy groups, C₁-C₆ alkyls or C₁-C₆ alkoxys.

In a further preferred embodiment, the acceptor is an open-chain aliphatic ketone, with the meaning of radical R² being a C₁-C₆ alkyl, carbonyl or —CH₂XCH₃ or —CHXCH₂CH₃ where X is a heteroatom, preferably sulphur or oxygen.

In another preferred embodiment, the acceptors are ketone having aromatic or heteroaromatic substituents where, in the compound having the formula

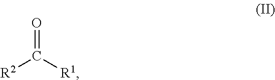

the meaning of R¹ is a C₁-C₆ alkyl and R² is a substituent having the formula -A-B-C-D where A and C are each —(CH₂)-ₙ where n=1 to 6 or are each a covalent bond and D is a monosubstituted or disubstituted aromatic or heteroaromatic radical as defined above.

In a preferred embodiment, R² is selected from:

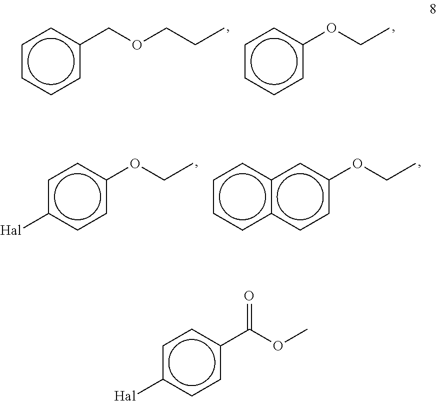

where Hal is selected from F, Cl, Br, or I.

In a further preferred embodiment, the substituents R¹ and R² may together form a cyclic alkyl radical which may possibly also have a heteroatom such as sulphur or oxygen. This cyclic alkyl radical preferably has 4-10 carbon atoms. As well as this, this cyclic alkyl radical may also be substituted with one or more C₁-C₆ alkyl radicals or heteroatoms, always provided that these are not 2-methylcyclohexanone, 3,3-dimethylcyclohexanone or CDP-activated 3,6-didesoxy-4-keto-D-glucose.

The acceptor substrates are preferably aldehydes, simple ketones, diketones, ketones having ether substituents or ketones having ester substituents. The general formulas and preferred examples are grouped together in the following overview table.

OVERVIEW TABLE 1
Range of acceptor substates for the enzyme YerE; here “R—”
means that the aromatic radical may possibly have one to four heteroatoms and/or the
C₁-C₆ alkyl may be monosubstituted or disubstituted.

| | Examples | |
|---|---|---|
| Aldehydes: | | |
| 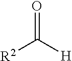 | 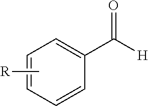 | |
| Simple ketones: | | |
| 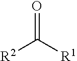 | 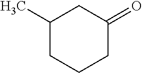 | |
| Diketones: | | |
| 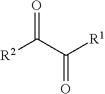 | 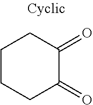 | 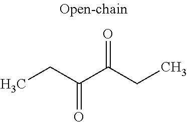 |
| Ketones having ester | | |
| substituents: | | |
| 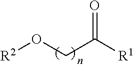 | 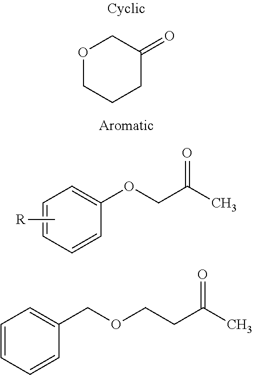 | 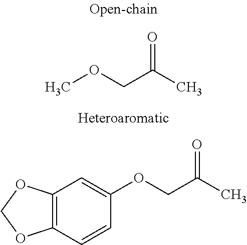 |
| Ketones having ester | | |
| substituents: | | |
| 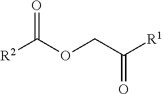 | 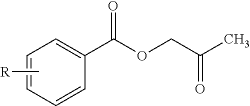 | |

Because the natural substrate of the enzyme YerE is pyruvate, pyruvate is a preferred donor substrate. However, as an alternative to this, the donor substrate appearing as a second reactand in the reaction equation (I) may be a compound having the formula:

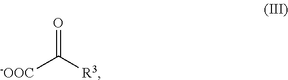

where the meaning of R³ is hydrogen or a C₁-C₆ alkyl.

With the thiamine diphosphate dependent enzyme YerE, it has been possible for the first time to show a catalytic asymmetrical C—C-bond linking of an activated (subjected to umpolung) aldehyde to simple ketones. At the same time, this is also the first example of an asymmetrical intermolecular variant of this reaction.

To date, there is no known example of the non-enzymatic asymmetrical intermolecular mixed benzoin condensation of an aldehyde with a ketone. Taking as a point of departure the physiological reaction of the enzyme which had been published in the literature, we have been able to show that asymmetrical induction by a chiral substrate is able to exceed the induction by the protein.

Only by using suitable prochiral ketones in conjunction with a 2-ketocarbonic acid as a starting substance for the forming of the activated aldehyde we were able to successfully catalyse the above reaction by YerE. The enantiomer-enriched tertiary alcohols to which access can thus be gained are an important group of building blocks for the pharmaceutical and chemical industries to which it would otherwise be difficult to gain access.

### EXAMPLE 1

### Preparation of the Enzyme YerE

### a) Cloning

The starting material for the work was genomic DNA from Yersinia pseudotuberculosis O:VI obtained from the Institute for Hygiene and Environment (Institut für Hygiene und Umwelt) of Hamburg (Abteilung für Mikrobiologie und Verbraucherschutz=Department of Microbiological Consumer Protection). As an alternative to this, what may also be obtained are suitable strains from collections of strains such as ATCC or DSM.

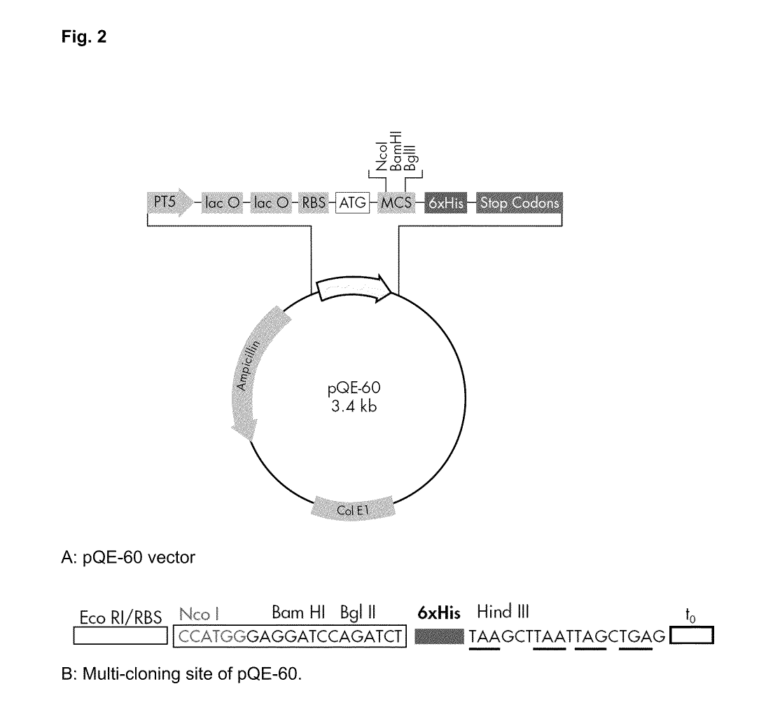

The DNA which coded for the protein YerE from Yersinia pseudotuberculosis O:VI was amplified by PCR. When this was done, an Ncol site was also inserted at the 5′ end and a BglII site at the 3′ end. The vector pQE-60 (FIG. 2, A) has these two sites at its multicloning site (MCS) (FIG. 2, B). Both the vector and the particular PCR product were then digested with the two restriction enzymes and following this the particular PCR product was ligated to the vector. With the construct obtained in this way, E. coli XL1blue was converted for vector production and E. coli BL21(DE3) cells for expression. A fusion protein in the form of a protein carrying a C-terminal His tag was obtained in this way from YerE from Yersinia pseudotuberculosis O:VI. The nucleotide sequence of YerE is shown in the sequence listing as SEQ ID NO:1.

### b) Expression

Expression took place in E. coli BL21(DE3) cells.

The cells were incubated in LB medium (100 μg/ml of ampicillin) at 37° C. in a shake flask and were expressed overnight at 24° C. following the addition of IPTG (1.0 mM).

### c) Purification of Protein

The purification of the protein was carried out by immobilised metal ion affinity chromatography. Both talon beads (cobalt ions) and nickel NTA were used.

Because of the very good expression of the protein YerE (protein content of approx. 5% in the raw extract) and the absence of side reactions, the raw extract, when lyophilised, was used in the case of the mixtures where ketones were the acceptor substrates and the case of the mixture where oxobutyrate was the donor substrate.

### EXAMPLE 2

### Pyruvate as a Substrate for YerE

If an electrophile (e.g. an aldehyde) or a further acceptor substrate is not present, YerE catalyses the condensation of pyruvate into acetoin (reaction equation IV). This reactivity has already been found in various pyruvate decarboxylases such for example as those from Zymomonas mobilis ((S)-acetoin) or Saccharomyces cerevisiae ((R)-acetoin).

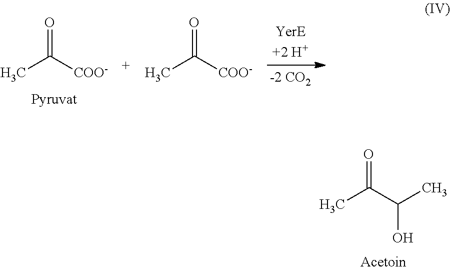

Key:

| German | English |
|---|---|
| Pyruvat | Pyruvate |

(IV) shows the YerE-catalysed conversion of pyruvate into acetoin. Reaction volume 1.5 ml, 50 mM of sodium pyruvate,

ee((S)-Acetoin)=4.3%, chiral GC (Lipodex, 70° C., Rₜ=13.6 min (R), 18.4 (S)).

### EXAMPLE 3

### Aldehydes as Acceptors

Benzaldehyde and Derivatives as Acceptors

Starting from pyruvate, (R)-1-phenyl-1-hydroxyketones were formed with YerE in the presence of aromatic aldehydes with good enantiomer selectivities (ee=80-97%), in accordance with reaction equation (V):

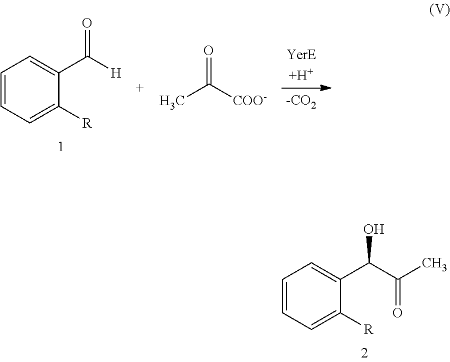

In the case of the educt (1), the substituents listed in Table 1 below were used for R.

TABLE 1

| | GC-MS | HPLC (OB column) |
|---|---|---|
| | conversion [%] | ee [%] |
| Product (2) | (after 16 h) | (after 16 h) |
| R = H | 82 | 97 |
| R = F | 98 | 94 |
| R = Cl | 97 | 94 |
| R = Br | 97 | 94 |
| R = I | 95 | 80 |
| R = CH₃ | 74 | 95 |

(V) shows the YerE-catalysed conversions when benzaldehyde and derivatives are used.

Reaction volume 1.5 ml, 20% DMSO, 20 mM of benzaldehyde derivative, 50 mM of sodium pyruvate, 300 μg of YerE protein.

This shows that even sterically hindered compounds such as 2-methyl benzaldehyde are good acceptors.

### EXAMPLE 4

### Cyclic Ketones as Acceptors

For the purified enzyme YerE, it could be shown that there is an intermolecular aldehyde-ketone benzoin reaction with cyclohexanone and methylated cyclohexanones by the decarboxylation of pyruvate (reaction equation VI).

TABLE 2
YerE-catalysed conversions of cyclohexanone and derivatives.
(VI)

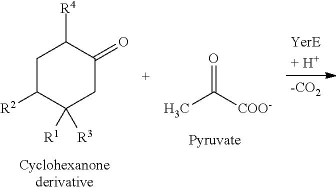

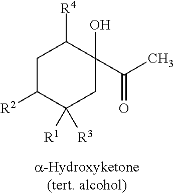

| | | | | | Conversion [%] |
|---|---|---|---|---|---|
| Educt | R¹ | R² | R³ | R⁴ | by GCMS |
| Cyclohexanone | H | H | H | H | 60.7 |
| 2-Methylcyclohexanone | H | H | H | CH₃ | — |
| 3-Methylcyclohexanone | CH₃ | H | H | H | 48.6 |
| (rac.) | | | | | |
| (R)-3-Methylcyclohexanone | CH₃ | H | H | H | 61.2 |
| 3,3-Dimethylcyclohexanone | CH₃ | H | CH₃ | H | — |
| 4-Methylcyclohexanone | H | CH₃ | H | H | 66.9 + 8.8 |
| | | | | | (2 isomers) |

Of a large number of compounds which were tested, only 2-methylcyclohexanone and 3,3-dimethylcyclohexanone were not converted by the enzyme YerE.

In the conversion of 3-methylcyclohexanone a chirality centre is created by the enzymatic reaction, and for this reason this conversion was picked out and examined in greater detail.

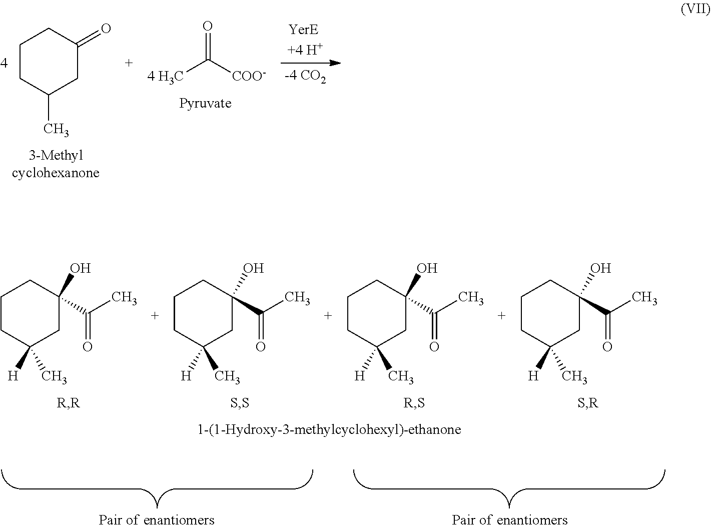

Reaction equation (VII): YerE-catalysed conversion of 3-methyl cyclohexanone (rac.) using pyruvate

The enzymatic product 1-(1-hydroxy-3-methylcyclohexyl)ethanone was synthesised chemically for comparative purposes. It was found that in the chemical reaction one of the two pairs of enantiomers is formed in a high excess compared with the other. Because of the fact that there exists, both for the R,R-diastereomer and for the S,S-diastereomer, a chair conformation which is beneficial from the point of view of energy and in which the two large substituents (the acetyl and methyl groups) occupy an equatorial position, whereas no such conformation exists for the R,S-diastereomer and the S,R-diastereomer, it can be assumed that the pair of enantiomers which are formed in excess are the R,R-diastereomer and S,S-diastereomer.

By NMR and GCMS examinations, all that were detected in the enzymatic conversion were the pair of enantiomers formed in a high excess by means of the chemical synthesis. Because an attempt to divide this pair of enantiomers by means of chiral GC did not produce the desired success, (S)-3-methyl cyclohexanone, which is not commercially available, was synthesised chemically by a single synthesising step to check whether both (R)-3-methyl cyclohexanone and (S)-3-methyl cyclohexanone are accepted as substrates by the enzyme YerE. When this was done it was found that the (S)-enantiomer too is converted by the protein into the corresponding diastereomer (VIII).

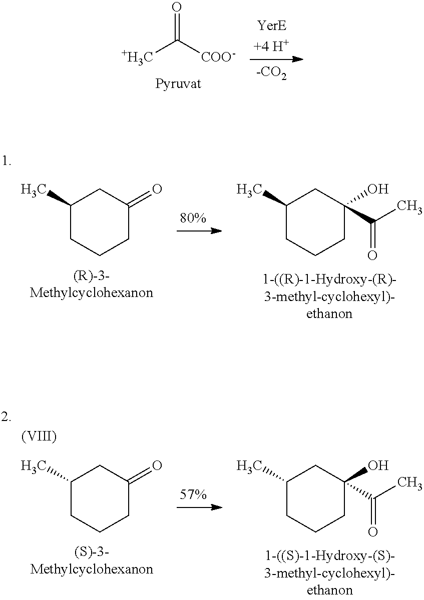

Key:

| German | English |
|---|---|
| Pyruvat | pyruvate |
| (R)-3-Methylcyclohexanon | (R)-3-Methylcyclohexanone |
| 1-((R)-1-Hydroxy-(R)-3-methyl- | 1-((R)-1-Hydroxy-(R)-3- |
| cyclohexyl)ethanon | methyl-cyclohexyl)ethanone |
| (S)-3-Methylcyclohexanon | (S)-3-Methylcyclohexanone |
| 1-((S)-1-Hydroxy-(R)-3-methyl- | 1-((S)-1-Hydroxy-(R)-3- |
| cyclohexyl)ethanon | methyl-cyclohexyl)ethanone |

(VIII): Product formation in % shown by GC-MS with cyclic aliphatic ketones as acceptor substrates. Reaction volume 1.5 ml, 20 mM of acceptor substrate, 50 mM of sodium pyruvate, 1. Total of 15.4 mg of protein used, 2. Total of 15.4 mg of protein used.

In the case of the conversion of 3-methyl cyclohexanone it is therefore suspected that it is kinetic and thermodynamic effects of the forming of the product and not the chiral information from the enzyme YerE which is crucial to the selectivity observed for the reaction. From this observation it can be concluded that, in the case of the physiological reaction of the protein (see FIG. 1), the setting-up of the stereocentre at C4 is likewise predetermined by the three substituents already bonded to the ring at C1, C2 and C5 rather than by the enzyme YerE.

### EXAMPLE 5

### Cyclic Aliphatic Ketones as Acceptors

It was found that, by decarboxylation by YerE catalysis, pyruvate is added to the cyclic aliphatic ketones cyclohexanone and 3-methyl cyclohexanone (reaction equation (IX)).

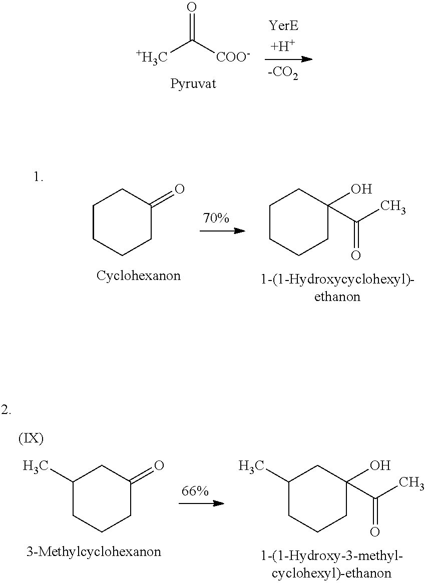

Key:

| German | English |
|---|---|
| Pyruvat | pyruvate |
| Cyclohexanon | Cyclohexanone |
| 1-(1- | 1-(1- |
| Hydroxycyclohexyl)ethanon | Hydroxycyclohexyl)ethanone |
| 3-Methylcyclohexanon | 3-Methylcyclohexanone |
| 1-(1-Hydroxy-3-methyl- | 1-(1-Hydroxy-3-methyl- |
| cyclohexy)ethanon | cyclohexy)ethanone |

(IX): Product formation in % shown by GC-MS with cyclic aliphatic ketones as acceptor substrates.

Reaction volume 1.5 ml, 20 mM of acceptor substrate, 50 mM of sodium pyruvate, 1. Total of 15.1 mg of protein used, 2. Total of 15.4 mg of protein used.

### EXAMPLE 6

### Cyclic Ketone, Having a Heteroatom, as the Acceptor

To enable any enzyme-catalysed asymmetrical reaction which there may be to be studied, the potential substrate tetrahydro-2H-pyran-3-one was synthesised by a two-stage chemical synthesis because in this case the stability of the product does not affect the reaction: in the present case, from the point of view of the thermodynamic aspects, it is equally probable for each of the two possible enantiomers to be formed.

By examination by GCMS, almost complete conversion of the substrate into the product shown, namely 1-(3-hydroxytetrahydro-2H-pyran-3-yl)ethanone (X), in the presence of pyruvate was detected.

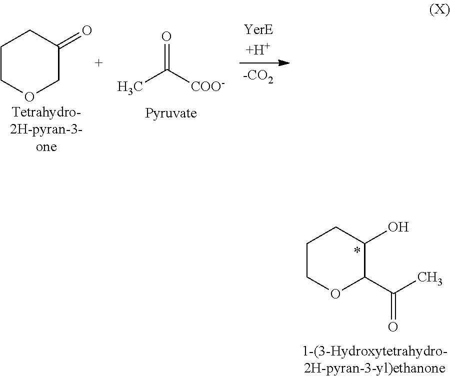

(X): YerE-catalysed conversion of tetrahydro-2H-pyran-3-one using pyruvate.

For preparation on the 50 ml scale, a total of 618 mg of protein was used and 97% of 1-(3-hydroxytetrahydro-2H-pyran-3-yl)ethanone was obtained, as given by simple NMR. After purification by column chromatography (R_{f}(dichloromethane/acetone=9:1)=0.32), the result was 49 mg of 1-(3-hydroxytetrahydro-2H-pyran-3-yl)ethanone (34% isolated yield)

GC-MS: t_{R}=6.53 min m/z=144 ([M]⁺; 1.2%), 126 ([M-H₂O]⁺; 15.6%), 101 ([C₅H₉O₂]⁺; 100%), 83 ([C₅H₇O]⁺; 26.1%), 55 ([C₄H₇]⁺; 65.0%).

¹H NMR: (400 MHz, CDCl₃, 25° C.): δ [ppm]=1.54-1.68 (m, 1H, CH₂), 1.71-1.79 (m, 1H, CH₂), 1.89-2.00 (m, 2H, CH₂), 2.32 (s, 3H, CH₃), 3.41 (s, 1H, OH), 3.47-3.72 (m, 3H, CH₂), 3.89-3.98 (m, 1H, CH₂).

¹³C NMR: (100 MHz, CDCl₃, 25° C.): δ [ppm]=21.1 (CH₂), 25.5 (CH₃), 31.3 (CH₂), 67.9 (CH₂), 72.6 (CH₂), 77.0 (Cq), 210.9 (C═O).

GC: (Lipodex, 90° C.) Rₜ=60.56 min (main enantiomer), 59.28 min (ent). ee=84% The enantiomeric excess was determined using the chemically synthesised racemate as a reference.

Specific rotation: [α]_{D}²²=+5.04 (α=+0.0564, c=1.1 g/100 ml, chloroform).

With the chemically synthesised racemate as a reference, an enantiomeric excess of 84% of the purified 1-(3-hydroxytetrahydro-2H-pyran-3-yl)ethanone was determined by chiral gas chromatography.

### EXAMPLE 7

### 1-Phenoxypropan-2-one as an Acceptor

Starting from the substrate tetrahydro-2H-pyran-3-one, it was possible by screening to identify the open-chain compound 1-phenoxypropan-2-one as a further substrate for the enzyme YerE.

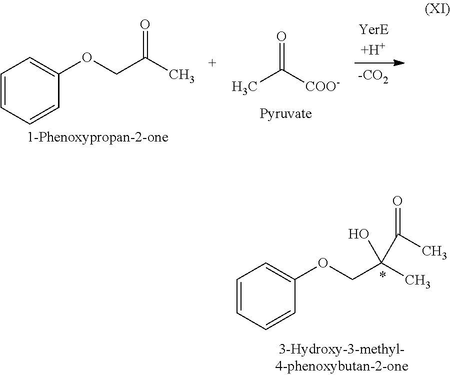

(XI): YerE-catalysed conversion of 1-phenoxypropan-2-one using pyruvate.

On the semi-preparative scale, 45% of 4.5 mg of 1-phenoxypropan-2-one was converted (GCMS) into 3-hydroxy-3-methyl-4-phenoxybutan-2-one using 14.9 mg of protein (≈745 μg of YerE protein).

For preparation on the 50 ml scale, a total of 588 mg of protein was used and 48% of 3-hydroxy-3-methyl-4-phenoxybutan-2-one was obtained, as given by simple NMR.

After purification by column chromatography (R_{f}(cyclohexane/ethyl acetate=5:1)=0.28), the result was 56 mg of 3-hydroxy-3-methyl-4-phenoxybutan-2-one (29% isolated yield)

GC-MS: t_{R}=9.26 min m/z=194 ([M]⁺; 11.6%), 151 ([M-C₂H₃O]⁺; 100%), 133 ([C₉H₉O]⁺; 64.0%), 94 ([C₆H₆O]⁺; 57.3%), 77 ([C₆H₅]⁺; 36.0%).

¹H NMR: (400 MHz, CDCl₃, 25° C.): δ [ppm]=1.47 (s, 3H, CH₃COH), 2.35 (s, 3H, CH₃C═O), 4.00 (s, 1H, OH), 4.02 (d, J=9.4 Hz, 1H, CH₂), 4.21 (d, J=9.4 Hz, 1H, CH₂), 6.85-6.93 (m, 2H, ar-H), 6.96-7.03 (m, 1H, ar-H), 7.25-7.34 (m, 2 H, ar-H).

¹³C NMR: (100 MHz, CDCl₃, 25° C.): δ [ppm]=21.7 (CH₃COH), 24.6 (CH₃C═O), 7.30 (CH₂), 78.3 (Cq), 114.6 (ar-C), 121.5 (ar-C), 129.5 (ar-C), 158.2 (ar-C), 210.3 (C═O).

HPLC: (Chiracel AS-H, 25° C., 0.75 ml·min⁻¹, n-hexane/2-propanol=90:10) Rₜ=10.15 min (ent) and 11.11 min (main enantiomer). ee=91%

Specific rotation: [α]_{D}²⁴=−43.7 (α=−0.5244, c=1.2 g/100 ml, chloroform).

The enantiomeric excess of 91% of the purified 3-hydroxy-3-methyl-4-phenoxybutan-2-one was determined by chiral HPLC with the chemically synthesised racemate as a reference.

### EXAMPLE 8

### Open-Chain Aliphatic Ketones as Acceptors

By YerE catalysis, pyruvate is also added to open-chain aliphatic ketones (equation XII).

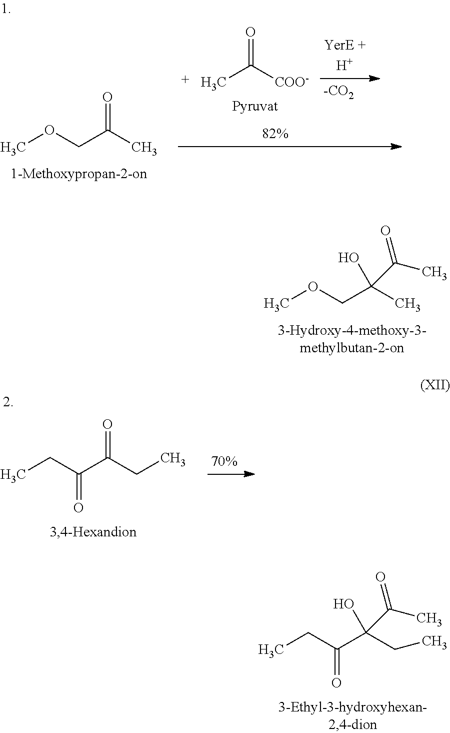

Key:

| German | English |
|---|---|
| Pyruvat | pyruvate |
| 1-Methoxypropan-2-on | 1-Methoxypropan-2-one |
| 3-Hydroxy-4-methoxy-3- | 3-Hydroxy-4-methoxy-3- |
| methylbutan-2-on | methylbutan-2-one |
| 3,4-Hexanedion | 3,4-Hexanedione |
| 3-Ethyl-3-hydroxyhexan-2,4- | 3-Ethyl-3-hydroxyhexane-2,4- |
| dion | dione |

(XII): Product formation in % as shown by GC-MS for open-chain aliphatic ketones as acceptor substrates. Reaction volume 1.5 ml, 20 mM of acceptor substrate, 50 mM of sodium pyruvate, 1. Total of 15.1 mg of protein used, 2. Total of 18.2 mg of protein used.

For preparation on the 50 ml scale, a total of 690 mg of protein was used. Because of the coincidence of the educt and product signals, it is not possible in the present case for a figure for the proportion by percent of 3-ethyl-3-hydroxyhexane-2,4-dione to be given from simple NMR.

After purification by column chromatography (R_{f}(cyclohexane/ethyl acetate=5:1)=0.50), the result was 53 mg of 3-ethyl-3-hydroxyhexane-2,4-dione (34% isolated yield)

GC-MS: t_{R}=5.71 min m/z=158 ([M]⁺; 0.6%), 116 ([C₆H₁₂O₂]⁺; 59.1%), 102 ([C₅H₁₀O₂]⁺; 99.8%), 87 ([C₄H₇O₂]⁺; 73.2%), 57 ([C₃H₅O]⁺; 100%). t_{R}=5.79 min m/z=158 (0.4%), 116 (9.5%), 102 (100%), 87 (52.2%), 57 (94.3%).

¹H NMR: (400 MHz, CDCl₃, 25° C.): δ [ppm]=0.83 (t, J=7.4 Hz, 3H, CH₃), 1.04 (t, J=7.3 Hz, 3H, CH₃), 2.03 (q, J=7.4 Hz, 2H, CH₂), 2.25 (s, 3H, CH₃), 2.44-2.60 (m, 1H, CH₂), 2.68-2.82 (m, 1H, CH₂), 4.66 (s, 1H, OH).

¹³C NMR: (100 MHz, CDCl₃, 25° C.): δ [ppm]=5.2 (CH₃), 5.2 (CH₃), 29.7 (CH₂), 30.9 (CH₂), 91.0 (Cq), 207.8 (C═O), 210.2 (C═O).

GC: (Lipodex D, 90° C.) Rₜ=10.68 min (main enantiomer), 12.02 min (ent). ee=84%

Specific rotation: [α]_{D}²²=−15.3 (α=−0.1778, c=1.2 g/100 ml, chloroform).

### EXAMPLE 9

### Ketones, Having Aromatic Substituents, as Acceptors

By YerE catalysis, pyruvate is also added to ketones having aromatic substituents (reaction equations XIII.1-XIII.5).

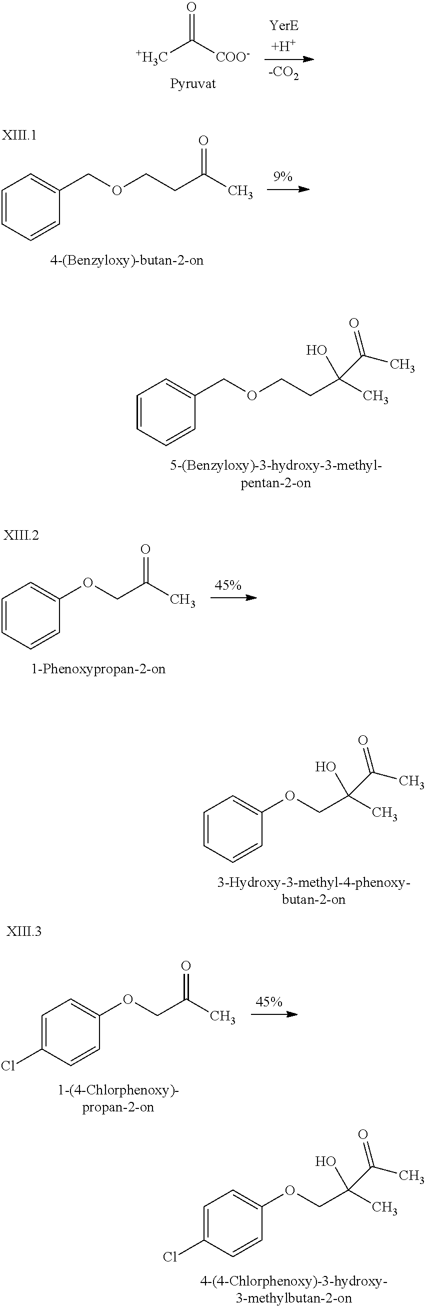

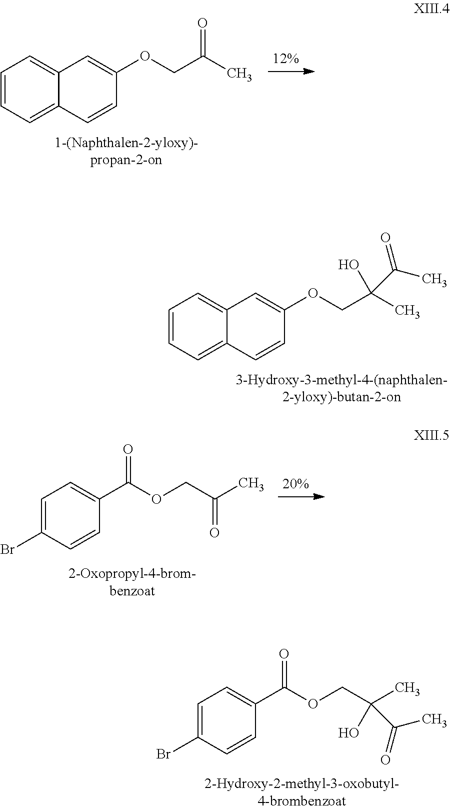

Key:

| German | English |
|---|---|
| Pyruvat | pyruvate |
| 4-(Benzyloxy)butan-2-on | 4-(Benzyloxy)butan-2-one |
| 5-(Benzyloxy)-3-hydroxy-3- | 5-(Benzyloxy)-3-hydroxy-3- |
| methyl-pentan-2-on | methyl-pentan-2-one |
| 1-Phenoxypropan-2-on | 1-Phenoxypropan-2-one |
| 3-Hydroxy-3-methylo-4-phenoxy- | 3-Hydroxy-3-methylo-4-phenoxy- |
| butan-2-on | butan-2-one |
| 1-(4-Chlorophenoxy)propan-2on | 1-(4-Chlorophenoxy)propan- |
| | 2one |
| 4-(4-Chlorophenoxy)-3-hydroxy- | 4-(4-Chlorophenoxy)-3-hydroxy- |
| 3-methylbutan-2-on | 3-methylbutan-2-one |
| 1-(Naphthalen-2-yloxy)propan-2- | 1-(Naphthalen-2-yloxy)propan-2- |
| on | one |
| 3-Hydroxy-3-methyl-4- | 3-Hydroxy-3-methyl-4- |
| (naphthalen-2-yloxy)-butan-2-on | (naphthalen-2-yloxy)-butan-2- |
| | one |
| 2-Oxopropyl-4-bromobenzoat | 2-Oxopropyl-4-bromobenzoate |
| -Hydroxy-2-methyl-3-oxobutyl-4- | -Hydroxy-2-methyl-3-oxobutyl-4- |
| bromobenzoat | bromobenzoate |

Equations XIII.1-XIII.5: Product formation in % as shown by GC-MS with aromatic ketones as acceptor substrates. Reaction volume 1.5 ml, 20 mM of acceptor substrate, 50 mM of sodium pyruvate, and, in the case of XIII.3, XIII.4 and XIII.5 20% DMSO.

XIII.1. Total of 18.3 mg of protein used, XIII.2. Total of 14.9 mg of protein used, XIII.3. Total of 17.5 mg of protein used, XIII.4. Total of 15.0 mg of protein used, XIII.5. Total of 18.3 mg of protein used.

### EXAMPLE 10

### Variation of the Donor Substrate

As a further donor substrate, 2-oxobutyrate shows activity in the presence of carbonyls.

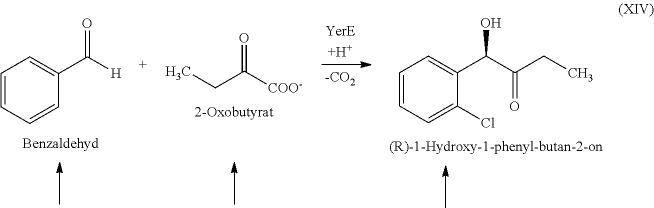

Key:

| German | English |
|---|---|
| Benzaldehyd | Benzaldehyde |
| 2-Oxobutyrat | 2-Oxobutyrate |
| (R)-1-Hydroxy-1-phenylbutan- | (R)-1-Hydroxy-1- |
| 2-on | phenylbutan-2-one |

Equation XIV: 2-Oxobutyrate as the donor substrate. Product formation from GC-MS: >99%.

Reaction volume 1.5 ml, 20 mM of acceptor substrate, 50 mM of 2-oxobutyrate, total of 19.7 mg of protein used ee((R)-1-Hydroxy-1-phenylbutan-2-on) >98%, chiral HPLC (Chiralcel OD-H, 40° C., 0.5 ml·min⁻¹, n-hexane/2-propanol=95:5, Rₜ=20.4 min (R)).

### EXAMPLE 11

### Reactions on the 50 ml Scale

### a) General Conditions of Preparation

20 mM of acceptor substrate and 50 mM of sodium pyruvate were used in each case. The protein lyophilisate was dissolved in 50 mM of KPi buffer. The acceptor substrate in pure form and the sodium pyruvate dissolved in 50 mM of Kpi buffer were added to this solution. In the enzymatic preparation process using 1-phenoxypropan-2-one, 2.5 ml of methyl tert-butyl ether (MTBE) (5%) was used in addition as a solubiliser. The reaction solution was then topped up to 50.0 ml with 50 mM of KPi buffer.

The protein lyophilisates contained 35-40% total protein used.

Composition of the KPi buffer:

50 mM KPi

1.5 mM MgCl₂

0.05 mM ThDP

0.02 mM FAD

### b) Processing

The reaction solution was extracted with methyl tert-butyl ether (MTBE) in the case of the preparations containing tetrahydro-2H-pyran-3-one and 3,4-hexanedione and with ethyl acetate in the case of the preparation containing 1-phenoxypropan-2-one. In the case of the mixture containing 3,4-hexandione, the reaction solution was first centrifuged to separate off the protein which had precipitated. The organic phase was dried over anhydrous sodium sulphate and the solvent was removed at reduced pressure.

The raw product was purified by column chromatography on Kieselgel 60. To purify the raw products obtained with tetrahydro-2H-pyran-3-one and 1-phenoxypropan-2-one as the starting materials, the purification was performed in Kieselgel 60 treated with hydrochloric acid.

### EXAMPLE 12

### Enantiomeric Excess

The enantiomeric excess of the products obtained by the YerE catalysis was determined by chiral GC-FID and chiral HPLC-DAD.

Enantiomeric excess of the products obtained by the YerE catalysis:

| 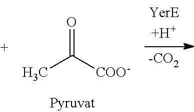 | | Enantiomeren- überschuss (ee %) |
|---|---|---|
| 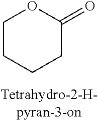 | 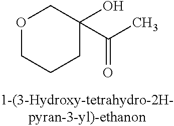 | 84% |
| 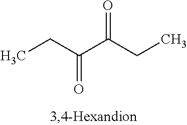 | 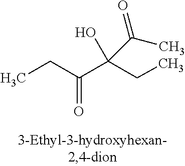 | 84% |
| 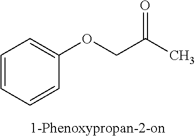 | 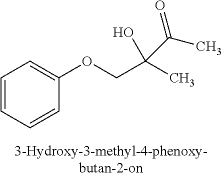 | 91% |

Key:

| German | English |
|---|---|
| Pyruvat | Pyruvate |
| Enantiomerenüberschuss (ee %) | Enantiomeric excess (ee %) |
| Tetrahydro-2-H-pyran-3-on | Tetrahydro-2-H-pyran-3-one |
| 1-(3-Hydroxytetrahydro-2H- | 1-(3-Hydroxytetrahydro-2H- |
| pyran-3-yl)ethanon | pyran-3-yl)ethanone |
| 3,4-Hexandion | 3,4-Hexanedione |
| 3-Ethyl-3-hydroxyhexan-2,4- | 3-Ethyl-3-hydroxyhexan-2,4- |
| dion | dione |
| 1-Phenoxypropan-2-on | 1-Phenoxypropan-2-one |
| 3-Hydroxy-3-methyl-4- | 3-Hydroxy-3-methyl-4- |
| phenoxybutan-2-on | phenoxybutan-2-one |

### EXAMPLE 13

### COMPARATIVE EXAMPLE

### a) Chemical Synthesis of racemic 1-(3-hydroxytetrahydro-2H-pyran-3-yl)ethanone

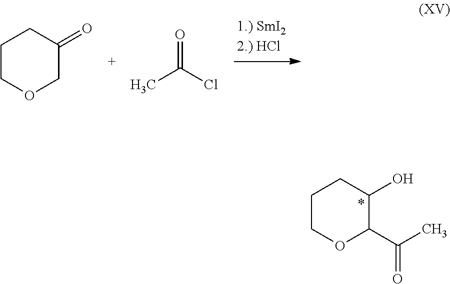

(XV): Chemical synthesis of racemic 1-(3-hydroxytetrahydro-2H-pyran-3-yl)ethanone as a reference.

GC-MS: t_{R}=6.53 min m/z=144 ([M]⁺; 1.2%), 126 ([M-H₂O]⁺; 15.6%), 101 ([C₅H₉O₂]⁺; 100%), 83 ([C₅H₇O]⁺; 26.1%), 55 ([C₄H₇]⁺; 65.0%).

¹H NMR: (400 MHz, CDCl₃, 25° C.): δ [ppm]=1.54-1.68 (m, 1H, CH₂), 1.71-1.79 (m, 1H, CH₂), 1.89-2.00 (m, 2H, CH₂), 2.32 (s, 3H, CH₃), 3.41 (s, 1H, OH), 3.47-3.72 (m, 3H, CH₂), 3.89-3.98 (m, 1H, CH₂).

¹³C NMR: (100 MHz, CDCl₃, 25° C.): δ [ppm]=21.1 (CH₂), 25.5 (CH₃), 31.3 (CH₂), 67.9 (CH₂), 72.6 (CH₂), 77.0 (Cq), 210.9 (C═O).

GC: (Lipodex, 90° C.) Rₜ=60.56 min, 59.28 min. Racemate

### b) Chemical synthesis of racemic 3-hydroxy-3-methyl-4-phenoxybutan-2-one

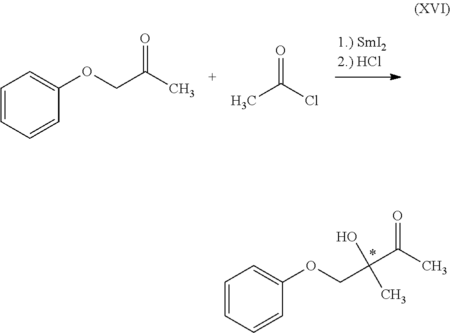

(XVI): Chemical synthesis of racemic 3-hydroxy-3-methyl-4-phenoxybutan-2-one as a reference.

GC-MS: t_{R}=9.26 min m/z=194 ([M]⁺; 11.6%), 151 ([M-C₂H₃O]⁺; 100%), 133 ([C₉H₉O]⁺; 64.0%), 94 ([C₆H₆O]⁺; 57.3%), 77 ([C₆H₅]⁺; 36.0%).

¹H NMR: (400 MHz, CDCl₃, 25° C.): δ [ppm]=1.47 (s, 3H, CH₃COH), 2.35 (s, 3H, CH₃C═O), 4.00 (s, 1H, OH), 4.02 (d, J=9.4 Hz, 1H, CH₂), 4.21 (d, J=9.4 Hz, 1H, CH₂), 6.85-6.93 (m, 2 H, ar-H), 6.96-7.03 (m, 1 H, ar-H), 7.25-7.34 (m, 2H, ar-H).

¹³C NMR: (100 MHz, CDCl₃, 25° C.): δ [ppm]=21.7 (CH₃COH), 24.6 (CH₃C═O), 73.0 (CH₂), 78.3 (Cq), 114.6 (ar-C), 121.5 (ar-C), 129.5 (ar-C), 158.2 (ar-C), 210.3 (C═O).

HPLC: (Chiracel AS-H, 25° C., 0.75 ml·min⁻¹, n-hexane/2-propanol=90:10) Rₜ=10.15 min and 11.11 min. Racemate

### EXAMPLE 14

### Absolute configurations

### a) Aldehydes as Acceptors

To determine the absolute configurations of the enzymatic products which were formed with benzaldehyde derivatives as starting materials, the conversion of 2-chlorobenzaldehyde (as an acceptor) with pyruvate (as a donor) was picked out and the product was isolated on the preparative scale.

### Synthesis of 1-(2-chlorophenyl)-1-hydroxypropan-2-one

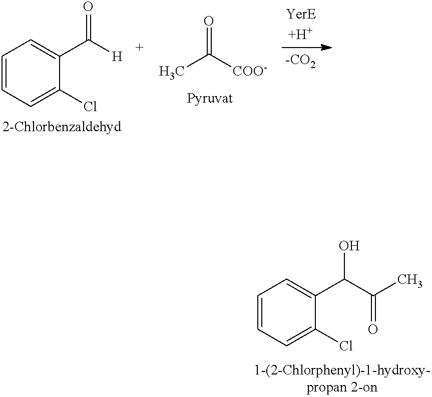

Key:

| German | English |
|---|---|
| 2-Chlorobenzaldehyd | 2-Chlorobenzaldehyde |
| Pyruvat | Pyruvate |
| 1-(2-chlorophenyl)-1- | 1-(2-chlorophenyl)-1- |
| hydroxypropan-2-on | hydroxypropan-2-one |

(XVII): YerE-catalysed conversion of 2-chlorobenzaldehyde to 1-(2-chlorophenyI)-1-hydroxypropan-2-one.

10 mg of YerE protein

90% 1-(2-chlorophenyl)-1-hydroxypropan-2-one as given by simple NMR

After purification by column chromatography

(R_{f}(cyclohexane/ethyl acetate=5:1)=0.25):

111 mg of 1-(2-chlorophenyl)-1-hydroxypropan-2-one (60% isolated yield)

GC-MS: t_{R}=8.80 min m/z=184 ([M]⁺; 0.13%), 143 ([C₆H₄ClCOH]⁺; 29%), 141 ([C₆H₄ClCOH]⁺; 100%), 77 ([C₆H₅]⁺; 76%), 51 ([C₄H₃]⁺; 12%).

¹H NMR: (400 MHz, CDCl₃, 25° C.): δ [ppm]=2.16 (s, 3H, CH₃), 4.37 (d, J=4.3 Hz, 1H, OH), 5.61 (d, J=4.3 Hz, 1H, CH), 7.29-7.33 (m, 3H, ar-H), 7.42-7.47 (m, 1H, ar-H).

¹³C NMR: (100 MHz, CDCl₃, 25° C.): δ [ppm]=25.3 (CH₃), 76.5 (CHOH), 127.5 (C-5′), 128.9, 129.9, 130.0 (C-3′,4′,6′), 133.4 (C_{q}), 135.6 (C_{q}), 206.3 (C═O).

HPLC: (Chiracel OB, n-hexane/2-propanol=90:10, 0.75 ml·min⁻¹, 20° C.) Rₜ=14.62 min (main enantiomer), Rₜ=12.58 min (ent). ee=73.9%

Specific rotation: [α]_{D}²³=−254.4 (α=−3.0913, c=1.2 g/100 ml, chloroform).

Molar CD: To determine the absolute configuration of the excess enantiomer, the CD spectrum of 1-(2-chlorophenyI)-1-hydroxy-propan-2-one was compared with that of (R)-phenylacetylcarbinol ((R)-PAC). For this purpose, the (R)-PAC was obtained by way of an enzymatic mixture using the enzyme pyruvate decarboxylase from Saccharomyces cerevisiae (ScPDC).

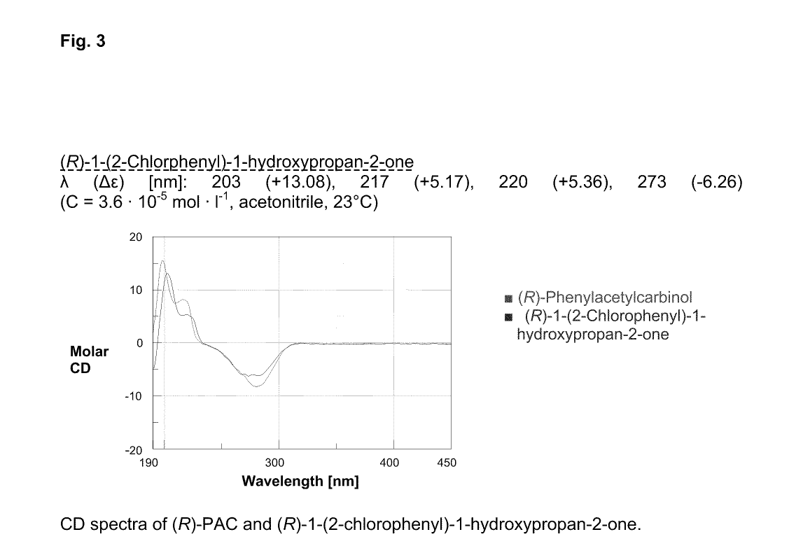

The CD spectrum is shown in FIG. 3.

Because (R)-PAC and the compound 1-(2-chlorophenyl)-1-hydroxypropan-2-one which was synthesised by YerE are molecules whose structures are very similar and which have comparable UV chromophores (aromatic hydrocarbon: π→π*, carbonyl function: n→π*), and which assume the same conformation, it can be concluded from the great similarity between the CD spectra of the two compounds (same minima and maxima) that the excess enantiomer in the synthesised compound is (R)-1-(2-chlorophenyI)-1-hydroxypropan-2-one.

### b) Ketones as Acceptors

At the present time nothing can be said about the absolute configuration of the enzymatic products shown which were obtained on the basis of ketones as acceptors because these are cases in which there is no data in the literature on appropriate reference substances which would allow the absolute configuration to be classified.

Optical Rotation

Specific rotation of the products obtained by YerE-catalysis:

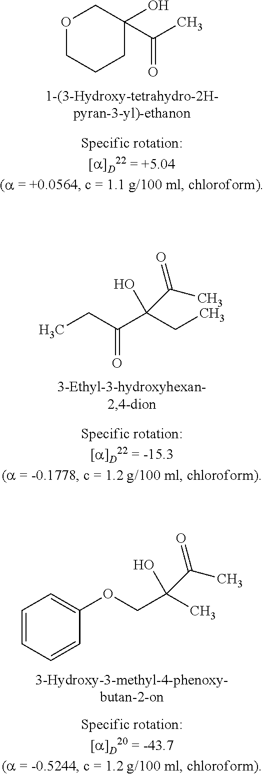

Key:

| German | English |
|---|---|
| 1-(3-Hydroxytetrahydro-2H- | 1-(3-Hydroxytetrahydro-2H- |
| pyran-3-yl)ethanon | pyran-3-yl)ethanone |
| 3-Ethyl-3-hydroxyhexan-2,4-dion | 3-Ethyl-3-hydroxyhexan-2,4- |
| | dione |
| 3-Hyrdoxy-3-methyl-4-phenoxy- | 3-Hyrdoxy-3-methyl-4-phenoxy- |
| butan-2-on | butan-2-one |

1) Method for forming a covalent bond between two groups having carbonyl radicals in accordance with the present reaction equation: 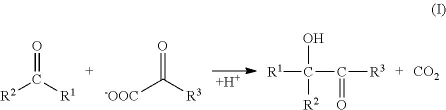
where
R¹ is hydrogen, a carboxyl group or a C₁-C₆ alkyl and
R² is an aromatic or heteroaromatic radical which may possibly be monosubstituted or disubstituted with a halogen and/or a C₁-C₆ alkyl or is a substituent having the formula -A-B-C-D where A and C are each a C₁-C₆ alkyl, carbonyl or a covalent bond, B is a heteroatom selected from oxygen and sulphur and D is an aliphatic radical having 1 to 6 carbon atoms, or an aromatic or heteroaromatic radical which may possibly be substituted,
or R¹ and R² together form a cyclic alkyl radical which may possibly be monosubstituted or disubstituted with a C₁-C₆ alkyl radical and/or with a hydroxy radical and which may possibly contain a heteroatom, namely oxygen or sulphur, and
R³ is hydrogen or a C₁-C₆ alkyl,
characterised in that the reaction is catalysed by the enzyme YerE.

2) Method according to claim 1, characterised in that the compound to be converted is not a cytidine-diphosphate-activated 3,6-didesoxy-4-keto-D-glucose.

3) Method according to claim 1 or 2, characterised in that the group having the formula 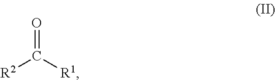
is, in the reaction equation (I), an acceptor in which the meaning of R¹ is hydrogen, a carboxyl group or a C₁-C₆ alkyl and R² is an aromatic radical which may possibly be monosubstituted or disubstituted with a heteroatom and/or a C₁-C₆ alkyl.

4) Method according to claim 1 or 2, characterised in that the group having the formula 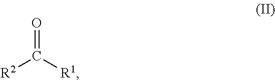
is, in the reaction equation (I), an acceptor in which the meaning of R¹ is a C₁-C₆ alkyl and R² is a substituent having the formula -A-B-C-D where the meaning of A and C is —(CH₂)-ₙ where n=1 to 6 or A and C are covalent bonds, B is a heteroatom selected from oxygen or sulphur or is a covalent bond, and D is a monosubstituted or disubstituted aromatic or heteroaromatic radical.

5) Method according to claim 4, characterised in that R² is selected from groups having the formulas: 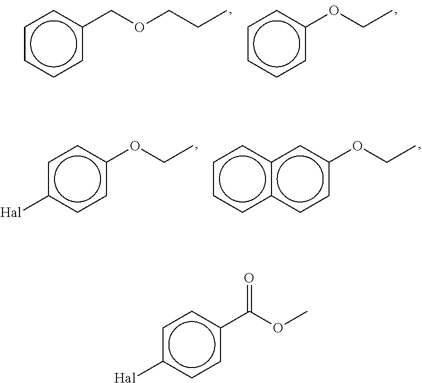
where Hal is selected from F, Cl, Br, or I.

6) Method according to one of the preceding claims, characterised in that the group having the formula 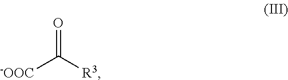
is a donor substrate in which the meaning of R³ is hydrogen or a C₁-C₆ alkyl.

7) Method according to one of the preceding claims, characterised in that the group having the formula (II) 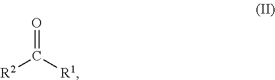
and the group having the formula (III) 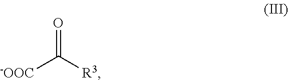
are not covalently connected to one another before the reaction in accordance with reaction equation (I).

8) Method according to one of claims 1-6, characterised in that the group having the formula (II) 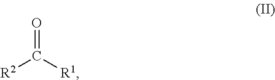
and the group having the formula (III) 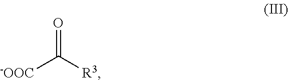
are covalently connected to one another even before the reaction in accordance with reaction equation (I) is carried out, with radical R¹ or radical R² being covalently connected to radical R³ and the covalent bonding in accordance with reaction equation (I) taking place intramolecularly.

9) Method according to one of the preceding claims, characterised in that the enzyme YerE is used in the form of a raw extract or a partially purified raw extract which contains at least 5% by weight of the enzyme YerE as a percentage of the total protein.

10) Method according to one of claims 1-8, characterised in that the enzyme YerE is used in the form of a purified enzyme preparation, characterised in that the enzyme preparation contains at least 80% by weight of YerE enzyme as a percentage of the total protein used.

11) Method according to one of the preceding claims, characterised in that the enzyme YerE is produced by recombinant methods in a host organism which does not belong to the genus Yersinia.

12) Method according to one of the preceding claims, characterised in that the enzyme YerE has an amino acid sequence which is at least 60% homologous to SEQ ID NO:2.

13) Use of an isolated enzyme YerE for forming a covalent carbon-carbon bond between two groups which each have a carbonyl group on the carbon atoms to be connected, the product which is produced in this case being produced with a high enantiomer ratio of >10:1.
